(19) United States
(12) Patent Application Publication
(10) Pub. No.: US 2025/0090742 A1
(43) Pub. Date: Mar. 20, 2025

(54) ENDOLUMINAL TREATMENT DEVICES AND RELATED METHODS

(71) Applicant: Boston Scientific Scimed, Inc., Maple Grove, MN (US)

(72) Inventors: Anthony Frank TASSONI, JR., Andover, MN (US); Martin BURKE, Galway (IE); Jason O'CONNELL, Co Galway (IE); Ryan WILBUR, County Galway (IE)

(73) Assignee: Boston Scientific Scimed, Inc., Maple Grove, MN (US)

(21) Appl. No.: 18/828,523

(22) Filed: Sep. 9, 2024

Related U.S. Application Data
(60) Provisional application No. 63/582,954, filed on Sep. 15, 2023.

(51) Int. Cl. A61M 1/00

(52) U.S. Cl. CPC A61M 1/96; A61M 2202/0014

(57) ABSTRACT

Medical devices and related methods are described, including a medical device that includes a tubular assembly including a plurality of tubes. Each tube of the plurality of tubes may be configured to be coupled to a vacuum source to deliver negative pressure to a distal end of each respective tube of the plurality of tubes. The medical device may further include a porous assembly including a plurality of sections and may be coupled to a distal end of the tubular assembly. Each section of the plurality of sections may be coupled to a respective tube of the plurality of tubes.

### CROSS-REFERENCE TO RELATED APPLICATIONS

This application claims the benefit of priority to U.S. Provisional Application No. 63/582,954, filed on Sep. 15, 2023, which is incorporated by reference herein in its entirety.

### TECHNICAL FIELD

Various aspects of this disclosure relate generally to minimally invasive medical devices and methods. In particular, aspects of the disclosure relate to medical devices and methods for endoscopic medical procedures, such as closing a wound or otherwise treating tissue

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical anastomotic leaks, or other wounds of the GI tract. Patients with perforations, post-surgical anastomotic leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices and methods of the current disclosure may rectify some of the deficiencies described above or address other aspects of the art.

### SUMMARY

According to some aspects of the present disclosure, a medical device may include a tubular assembly including a plurality of tubes. Each tube of the plurality of tubes may be configured to be coupled to a vacuum source to deliver negative pressure to a distal end of each respective tube of the plurality of tubes. The medical device may further include a porous assembly including a plurality of sections. The porous assembly may be coupled to a distal end of the tubular assembly, and each section of the plurality of sections may be coupled to a respective tube of the plurality of tubes.

According to some aspects, each tube of the plurality of tubes may include one or more walls defining a channel to deliver negative pressure to its respective section of the plurality of sections. In some examples, a first channel of an innermost tube of the plurality of tubes may extend from a proximal end to a distal end of the innermost tube. In some examples, a central longitudinal axis of the medical device may extend through the first channel. In some examples, a second channel of an outermost tube of the plurality of tubes may extend from a proximal end to a distal end of the outermost tube of the plurality of tubes. In some examples, the second channel extends radially around the central longitudinal axis of the medical device. In some examples, each section of the plurality of sections may include openings in fluid communication with the channel of the respective tube of the plurality of tubes, and the openings of each section of the plurality of sections may be independent of the openings of other sections of the plurality of sections. In some examples, the channel of each of the plurality of tubes may have an open proximal end and an open distal end, and the open proximal end of the channel may be coupled to the vacuum source and the open distal end may be coupled to a proximal end of a respective section of the plurality of sections. In some examples, the plurality of sections may be configured to be removed individually from a target site within a body lumen. In some examples, an outermost tube of the plurality of tubes may be coupled to an outermost section of the plurality of sections, and proximal movement of the outermost tube relative to other tubes of the plurality of tubes may retract the outermost section of the plurality of sections over a remainder of the plurality of sections. In some examples, the plurality of tubes may be configured to deliver negative pressure to only one section of the plurality of sections at a time. In some examples, each section of the plurality of sections may include an inner surface and an outer surface, and the outer surface of each section of the plurality of sections may include one or more of a course portion to collect exudates from a target site or a coating to prevent tissue ingrowth. In some examples, each section of the plurality of sections may include a cap at a distalmost face of each section of the plurality of sections. In some examples, a diameter of a distal end of each tube of the plurality of tubes may align with a diameter of a proximal end of a respective section of the plurality of sections. In some examples, the porous assembly may include at least four sections and the tubular assembly may include at least four tubes, and a first section may be coupled to a first tube, a second section may be coupled to a second tube, a third section may be coupled to a third tube, and a fourth section may be coupled to a fourth tube. In some examples, the first section may be cylindrical, the second section may extend around the first section, the third section may extend around the second section, and the fourth section may extend around the third section. In some examples, the first tube may be nested within the second tube, the second tube may be nested within the third tube, and the third tube may be nested within the fourth tube.

According to some aspects of the present disclosure, a medical device may include a tubular assembly including at least two tubes. Each tube of the tubular assembly may include a channel and each channel may be configured to be coupled to a vacuum source to deliver negative pressure to a distal end of each tube independently of other channels. The medical device may further include a porous assembly including at least two sections and coupled to a distal end of the tubular assembly. Each section of the porous assembly may be coupled to a respective channel of the at least two tubes. In some examples, an outermost tube of the at least two tubes may be coupled to an outermost section of the at least two sections, and proximal movement of the outermost tube relative to an innermost tube of the at least two tubes may move the outermost section of the at least two sections proximally over an innermost section of the at least two sections and over the innermost tube of the at least two tubes. In some examples, a channel of the innermost tube of the at least two tubes may be cylindrical, and a channel of the outermost tube may extend radially around a central longitudinal axis of the medical device.

According to some aspects of the present disclosure, a medical device may include a tubular assembly including a plurality of tubes nested within one another. Each tube of the plurality of tubes may be configured to be coupled to a vacuum source to deliver negative pressure to a distal end of each tube. The medical device may further include a porous assembly including a plurality of sections nested within one another and coupled to a distal end of the tubular assembly. A proximal end of each section of the plurality of sections may be coupled to a distal end of a respective tube of the tubular assembly. In some examples, each tube of the plurality of tubes may be configured to independently deliver negative pressure to a respective section of the plurality of sections.

### BRIEF DESCRIPTION OF THE DRAWINGS

The accompanying drawings, which are incorporated in and constitute a part of this specification, illustrate various aspects of this disclosure and together with the description, serve to explain the principles of the disclosure.

FIG. 1A depicts a distal end portion of an exemplary medical device in a first stage of usage in a target site, in accordance with some aspects of the present disclosure.

FIG. 1B is an enlarged view of a section of the distal end portion of the medical device of FIG. 1A, in accordance with some aspects of the present disclosure.

FIG. 1C depicts a distal end of the medical device of FIG. 1A, in accordance with some aspects of the present disclosure.

FIG. 1D depicts a proximal end of the medical device of FIG. 1A, in accordance with some aspects of the present disclosure.

FIG. 1E depicts the distal end portion of the medical device in FIG. 1A in a second stage of usage in the target site, in accordance with some aspects of the present disclosure.

FIG. 1F depicts the distal end portion of the medical device in FIG. 1A in a third stage of usage in the target site, in accordance with some aspects of the present disclosure.

FIG. 1G depicts an exemplary handle of the medical device in FIG. 1A, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a nasogastric tube having a sponge-like material (e.g., Granufoam™ sponge) or foam (e.g., vacuum sealed foam) sutured at its distal end. A proximal end of the tube may be connected to a collection container. The foam is placed endoscopically into the perforation, leak, or other wound. In some examples, EVAC includes endoluminal placement of a foam or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine.

Rat-tooth forceps or another accessory device may be extended through a working channel of the scope and used to guide the foam to the wound site as the scope is navigated to the wound site. Placement of the material can also be in other organs reachable via the GI tract (e.g., the colon). Negative pressure then is applied. The foam in the wound, along with the negative pressure, may accelerate healing by encouraging local tissue granulation at a wound site. The foam may be replaced with increasing smaller sizes of foam as the wound heals and closes. Present devices and systems suited for EVAC are limited. For example, EVAC typically requires a foam to be replaced every 3 to 5 days to prevent tissue ingrowth.

Aspects of this disclosure include devices and methods to reduce the number of foam exchanges and/or eliminate the need for foam exchanges and/or device exchanges during EVAC. Components of the devices described herein may be packaged as a kit for EVAC. For example, the devices disclosed herein may include a porous assembly coupled to a distal end of a tubular assembly. The porous assembly may include a plurality of porous layers or porous sections independent of one another. In some aspects, each porous layer or porous section may include a coating to prevent tissue ingrowth, an end cap, and/or a coarse portion on an outer surface of each porous layer or porous section to store and/or to collect exudates from a target site. In some aspects, the tubular assembly may include multiple coaxial tubes and each tube may be coupled to a respective porous layer or porous section. In some aspects, each tube may include a channel configured to deliver negative pressure to its respective porous layer or porous section independent or separate of the other channels. In some aspects, each porous layer or porous section may be removed from a target site sequentially, starting with an outermost porous layer or porous section by retracting its respective tube.

Reference will now be made in detail to examples of the present disclosure described above and illustrated in the accompanying drawings. Wherever possible, the same reference numbers will be used throughout the drawings to refer to the same or like parts.

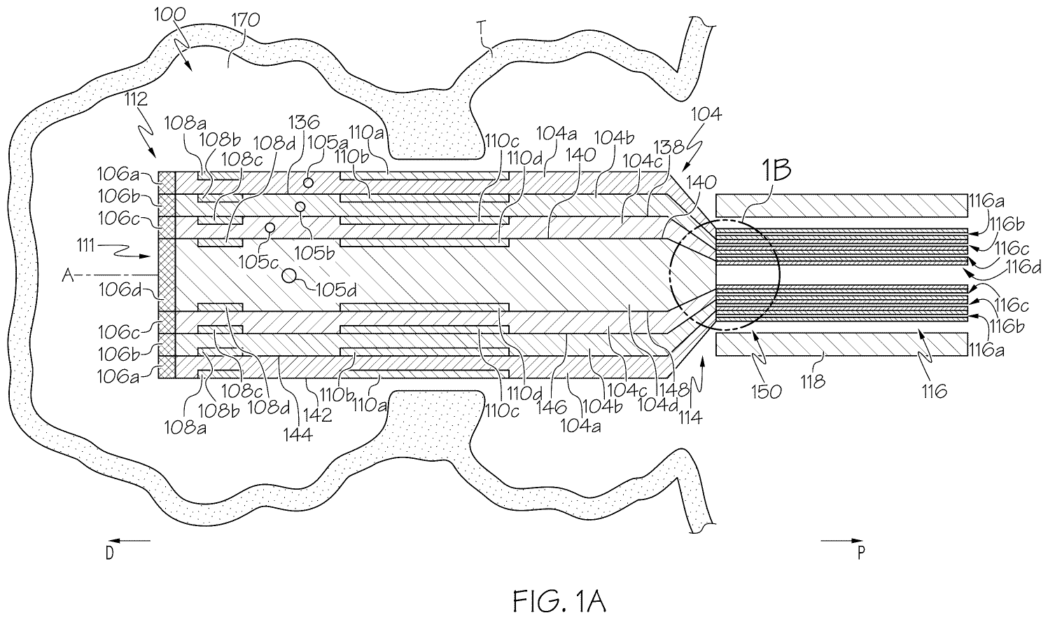

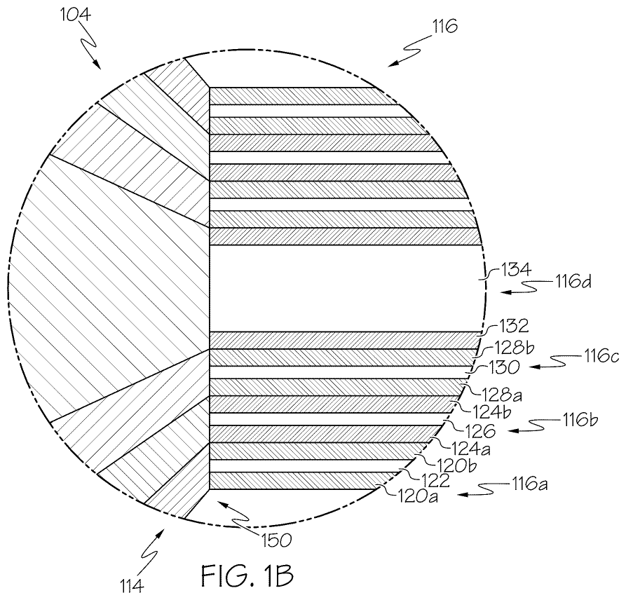

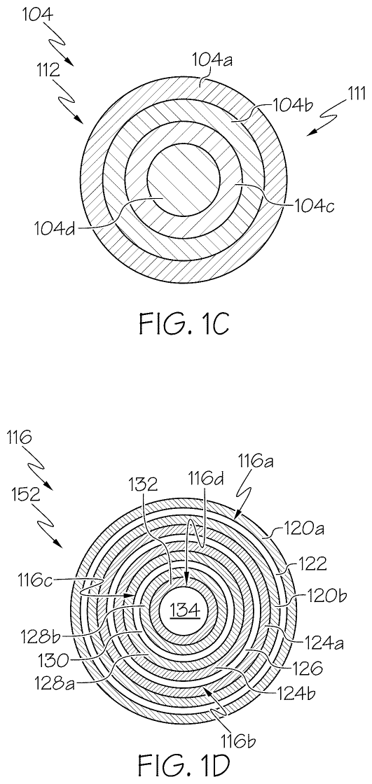

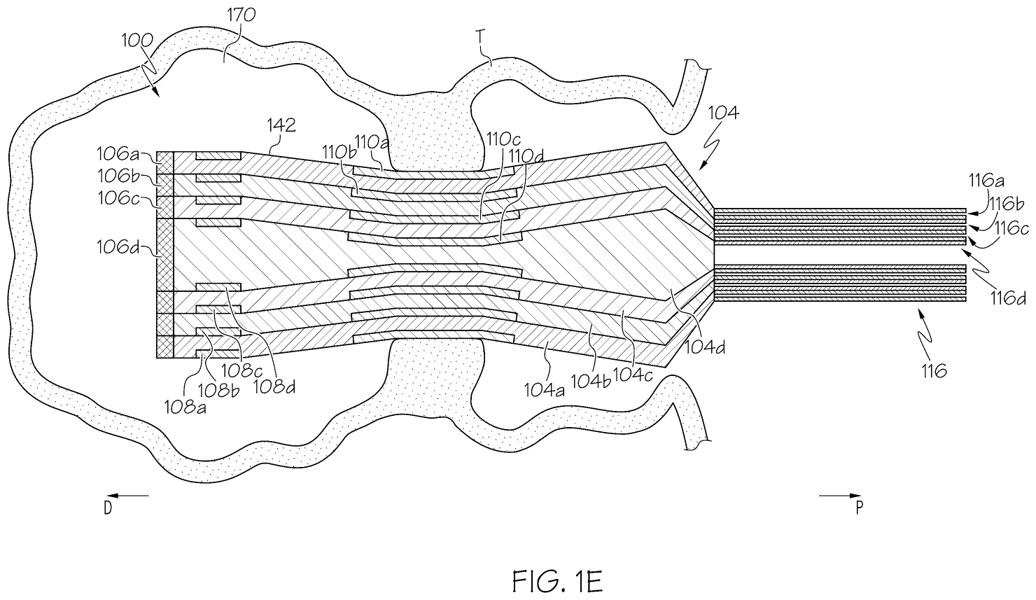

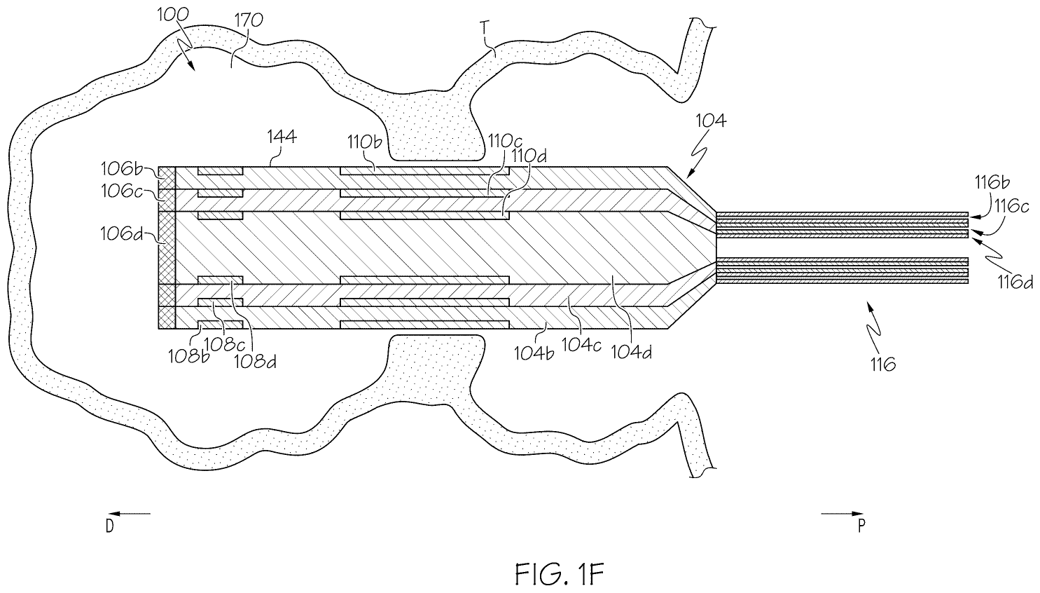

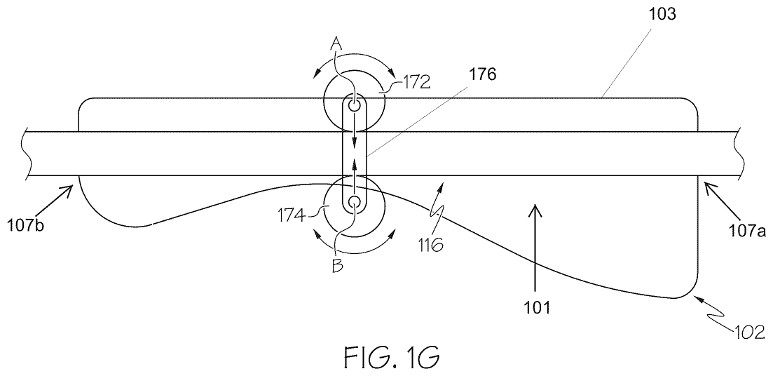

FIG. 1A illustrates a cross-sectional view of a distal portion of an EVAC device 100 including a porous or foam assembly 104, that may be inserted into a patient to help the healing of a wound, an anastomosis, or the like, or to otherwise treat tissue, as described above. Although EVAC device 100 is shown at a wound site or target site 170 (e.g., an anastomosis) in the GI tract, it will be appreciated that use of EVAC device 100 is not limited to the GI tract and may be used in any internal body lumen to treat wounds, leaks, perforations, etc. Foam assembly 104 may be substantially cylindrical and coupled to a tubular assembly 116. A central longitudinal axis A of EVAC device 100 may be aligned with a central longitudinal axis of foam assembly 104 and a central longitudinal axis of tubular assembly 116 (both also shown as axis A in FIG. 1A). Foam assembly 104 may include a plurality of foam layers or foam sections 104d, 104c, 104b, 104a.

FIG. 1C illustrates a distalmost face 111 of a distal end 112 of foam assembly 104 without end caps 106d, 106c, 106b, 106a, which will be described in greater detail below. A core foam or central section 104d may be substantially cylindrical (e.g., a solid cylinder) and each of the other sections 104c, 104b, 104a may extend radially around central section 104d forming coaxial, substantially tubular cylindrical layers or sections 104c, 104b, 104a that are nested within one another. As will be discussed in greater detail below, sections 104a, 104b, 104c, 104d may be removed from target site 170 one at a time during a course of a treatment and as target site 170 heals (i.e., becomes smaller). Thus, a diameter of foam assembly 104 may decrease as sections 104a, 104b, and/or 104c are removed to accommodate a size of a wound at varying stages of healing.

Each section 104d, 104c, 104b, 104a may extend longitudinally in the direction of central longitudinal axis A. In a cross-section that is perpendicular to central longitudinal axis A, section 104d may have a solid circular shape, while sections 104a, 104b, and 104c may have annular shapes. In some examples, sections 104d, 104c, 104b, 104a may have a same thickness measured in a direction perpendicular to central longitudinal axis A (i.e., a radial direction), and sections 104d, 104c, 104b, 104a may have a same length measured parallel to central longitudinal axis A (i.e., an axial direction). In other examples, sections 104d, 104c, 104b, 104a may have different thicknesses and/or lengths. Radially inner surfaces of each section 104c, 104b, 104a may contact a radially outer surface of the adjacent section 104d, 104c, 104b, respectively when the adjacent section is present (not removed).

Each section 104d, 104c, 104b, 104a may be independent of one another and each section 104c, 104b, 104a may be configured to be movable over a remainder of sections of foam assembly 104. Each section 104c, 104b, 104a may be configured to be movable over/along and relative to one or more of sections 104d, 104b, 104c. For example, section 104a may be configured to move proximally over and relative to sections 104b, 104c, 104d; section 104b may be configured to move proximally over and relative to sections 104c, 104d; and section 104c may be configured to move proximally over and relative to section 104d. This may allow for the removal of sections 104d, 104c, 104b, 104a from target site 170 over a course of a treatment sequentially, starting with an outermost layer or section (e.g., layer or section 104a) from central longitudinal axis A. In alternatives, other removal techniques may be utilized (e.g., one or more of sections 104a, 104b, and/or 104c may include perforations or other features to facilitate removal). For example, section 104a may be removed at day 4, section 104b may be removed at day 8, section 104c may be removed at day 12, and section 104d may be removed at day 16. The timeline provided above is merely exemplary, and any suitable timeline (including a customized patient timeline) may be utilized.

The size of foam assembly 104, including sections 104d, 104c, 104b, 104a, may depend on, e.g., the size of a wound or target site. Although EVAC device 100 is illustrated as having four sections 104d, 104c, 104b, 104a, it will be appreciated that foam assembly 104 may include any number of sections or layers, e.g., at a start of a treatment, and the number of sections or layers may depend on, e.g., an expected length of the treatment and/or size of the wound or target site. An operator may optionally remove one or more of sections 104a, 104b, 104c in order to customize a size of foam assembly 104 before initial delivery. It will be appreciated that foam assembly 104, including sections 104d, 104c, 104b, 104a, may be any shape, including spherical, cuboidal, irregular or the like.

Sections 104d, 104c, 104b, 104a may have any features of any foam that is known in the art for use in EVAC procedures. For example, sections 104d, 104c, 104b, 104a may include an open-cell foam. Sections 104d, 104c, 104b, 104a may include openings 105d, 105c, 105b, 105a, respectively, on an outer surface and/or interior thereof. Although FIG. 1A depicts each section 104d, 104c, 104b, 104a as having only one opening 105d, 105c, 105b, 105a for ease of illustration, it will be appreciated that each section 104d, 104c, 104b, 104a may include numerous openings 105d, 105c, 105b, 105a of any suitable shape, size, or location. Openings 105d, 105c, 105b, 105a may be any hole, pore, or channel. Openings 105d, 105c, 105b, 105a may include interconnecting channels and/or pores throughout sections 104d, 104c, 104b, 104a, respectively. For example, openings 105d, 105c, 105b, 105a may be pores of sections 104d, 104c, 104b, 104a, respectively. Openings 105d, 105c, 105b, 105a may have different sizes and/or shapes. Features of openings 105d, 105c, 105b, 105a (e.g., a size and shape of pores of sections 104d, 104c, 104b, 104a) may be selected based on a location of treatment within the body, properties of a wound to be treated, a stage of treatment, or other factors.

Sections 104d, 104c, 104b, 104a may include any suitable biocompatible material that may absorb fluids and/or permit fluid or other materials to pass therethrough via, e.g., negative pressure applied to sections 104d, 104c, 104b, 104a. The material of sections 104d, 104c, 104b, 104a may be flexible, compressible, porous, hydrophilic, sterile, and/or disposable. Suitable materials include polyurethanes, esters, ethers, composite materials, and/or other medical-grade materials.

Inner surfaces 140, 138, 136 of sections 104c, 104b, 104a, respectively, as shown in FIG. 1A, may be coated with a material and/or include a barrier to prevent fluids and/or other materials from target site 170 from passing through an outermost section, e.g., section 104a, and into inner sections, e.g., sections 104b, 104c, 104d, when, e.g., negative pressure is applied to the outermost section, e.g., section 104a. The material may include polytetrafluoroethylene (PTFE), expanded PTFE (ePTFE), high density polyethylene (HDPE), and/or thermoplastic elastomer (TPE). The coating material on inner surfaces 140, 138, 136 of sections 104c, 104b, 104a may isolate sections 104d, 104c, 104b, 104a from one another, including openings 105d, 105c, 105b, 105a. Outer surfaces 148, 146, 144, 142 of sections 104d, 104c, 104b, 104a may allow for fluids and/or other materials from target site 170 to pass through sections 104d, 104c, 104b, 104a when outer surfaces 148, 146, 144, 142 of sections 104d, 104c, 104b, 104a, respectively, are exposed to target site 170. For example, when section 104a is removed from target site 170, outer surface 144 of section 104b may be exposed to target site 170 and may allow for fluids and/or materials from target site 170 to pass through section 104b (as shown in FIG. 1F), but fluids and/or materials may not pass through sections 104c, 104d due to the coating material on inner surface 138 of section 104b.

Each section 104a, 104b, 104c, 104d may include an end cap 106a, 106b, 106c, 106d, a coarse portion 108a, 108b, 108c, 108d, and/or a foam coating 110a, 110b, 110c, 110d, respectively. Although reference will now be made to end cap 106a, coarse portion 108a, and foam coating 110a, it will be appreciated that end caps 106b, 106c, 106d may have the same or similar properties to end cap 106a; coarse portions 108b, 108c, 108d may have the same or similar properties to coarse portions 108a; and foam coatings 110b, 110c, 110d may have the same or similar properties to foam coating 110a.

End cap 106a may be removably coupled to a distalmost face of section 104a. For example, end cap 106a may be attached to the distalmost face of section 104a via adhesive. A material of end cap 106a may include ePTFE, PTFE, HDPE, and/or TPE. End cap 106a may assist with trapping fluids and/or material from target site 170 within section 104a during removal of section 104a from target site 170. For example, end cap 106a may prevent fluids and/or material within section 104a from leaking out of the distalmost face of section 104a during removal of section 104a from target site 170. End cap 106a may also prevent fluids and/or material from target site 170 from entering section 104a from the distalmost face of section 104a.

Outer surface 142 of section 104a may include coarse portion 108a at a distal end of section 104a or at another suitable location of section 104a. Coarse portion 108a may be configured to collect and/or store exudates from target site 170. Coarse portion 108a of outer surface 142 may extend entirely around a circumference of section 104a (e.g., form a ring-like coarse portion 108a on outer surface 142), or partially extend around a circumference of section 104a (e.g., form a patch-like coarse portion 108a). Although coarse portion 108a is illustrated at the distal end of section 104a, it will be appreciated that coarse portion 108a may be positioned anywhere along outer surface 142 (or other portion) of section 104a.

Outer surface 142 of section 104a may include foam coating 110a at a central portion of section 104a. Foam coating 110a of outer surface 142 may extend entirely around a circumference of section 104a, e.g., form a ring-like foam coating 110a on outer surface 142. Alternatively, foam coating 110a may extend only partially around section 104a (similar to a patch). Although foam coating 110a is illustrated at the central portion of section 104a, it will be appreciated that foam coating 110a may be positioned anywhere along outer surface 142 of section 104a. Foam coating 110a may be configured to prevent or inhibit tissue ingrowth into sections 104a. A material of foam coating 110a may include PTFE, silicon, polyvinylidene fluoride (PVDF), and/or HDPE. For example, as shown in FIG. 1E, as target site 170 becomes smaller (i.e., as the wound heals), tissue T at target site 170 may contact outer surface 142 of section 104a and compress sections 104a, 104b, 104c, 104d, but may not grow into sections 104a, 104b, 104c, 104d due to foam coating 110a.

FIG. 1B is an enlarged cross-sectional view of a proximal end 114 of foam assembly 104 and a distal end 150 of tubular assembly 116. Proximal end 114 of foam assembly 104 may be coupled to a distal end 150 of tubular assembly 116. Tubular assembly 116 may include a plurality of coaxial tubes 116a, 116b, 116c, 116d that are nested within one another. For example, tube 116a (e.g., an outermost tube) may surround tubes 116b, 116c, 116d, tube 116b may surround tubes 116c, 116d, and tube 116c may surround tube 116d (e.g., an innermost tube). Tubular assembly 116 may be and/or form, e.g., a nasogastric tube. In examples, each tube 116a, 116b, 116c, 116d may be thin-walled and comprised of a coil and polymer cover. Alternatively, tubes 116a, 116b, 116c, 116d may have any other suitable properties and may be constructed of any suitable material(s).

Tubes 116a, 116b, 116c, 116d may be fixedly coupled to sections 104a, 104b, 104c, 104d, respectively. Proximal ends of sections 104a, 104b, 104c, 104d may be attached to distal ends of tubes 116a, 116b, 116c, 116d, respectively, via sutures or other ties, an adhesive, a shrink-wrapped material, elastic(s), or the like. In a relaxed configuration of foam assembly 104, proximal end 114 of foam assembly 104 may be narrower than distal end 112 of foam assembly 104 to assist with, e.g., the coupling of proximal ends of sections 104a, 104b, 104c, 104d to distal ends of tubes 116a, 116b, 116c, 116d, respectively. A diameter of a distal end of each tube 116a, 116b, 116c, 116d may correspond to or otherwise align with a diameter of a proximal end of each section 104a, 104b, 104c, 104d to form one to one pairings/couplings between tubes 116a, 116b, 116c, 116d and sections 104a, 104b, 104c, 104d. In some examples, foam assembly 104 may be compressed (e.g., within a delivery sheath) during delivery, such that distal end 112 has a similar diameter to proximal end 114 during delivery.

Each tube 116a, 116b, 116c, 116d may be configured to deliver negative pressure independent of one another via channels within each tube 116a, 116b, 116c, 116d. As shown in FIG. 1B, distal ends of tubes 116a, 116b, 116c, 116d may align with proximal ends of sections 104a, 104b, 104c, 104d, respectively, which may allow for negative pressure to be applied to sections 104a, 104b, 104c, 104d individually. For example, tube 116a may apply negative pressure to section 104a, tube 116b may apply negative pressure to section 104b, tube 116c may apply negative pressure to section 104c, and tube 116d may apply negative pressure to section 104d.

FIG. 1D illustrates a proximal end 152 of tubular assembly 116. Tube 116a may include an outer wall 120a, an inner wall 120b, and a lumen/channel 122 disposed between walls 120a, 120b. Tube 116a may thus be comprised of two tubular walls 120a, 120b, which may be concentric with one another (e.g., about central longitudinal axis A). Channel 122 extends from a proximal end 152 of tube 116a, shown in FIG. 1D, to distal end 150 of tube 116a, shown in FIG. 1B. Channel 122 may extend radially around central longitudinal axis A of EVAC device 100. A cross-section of channel 122 that is perpendicular to central longitudinal axis A may have an annular (ring) shape. Channel 122 may have open proximal and distal ends. The open proximal end may be coupled to a vacuum source (not shown) and the open distal end may be coupled to the proximal end of section 104a. Openings 105a of section 104a may thus be in fluid communication with the open distal end of channel 122 of tube 116a. The vacuum source may supply a negative pressure to section 104a via the open distal end of channel 122. For example, a negative pressure of approximately 125 mm Hg, or approximately 2.5 pounds per square inch (PSI), may be supplied to section 104a via channel 122 of tube 116a. Other suitable amounts of negative pressure may be used. The negative pressure may pull fluid, material, and/or other debris into channel 122 of tube 116a via openings 105a of section 104a, which may promote healing of target site 170.

Tubes 116b and 116c may have similar properties to tube 116a, unless stated otherwise herein. Tube 116b may include an outer wall 124a, an inner wall 124b, and a cylindrical lumen/channel 126 disposed between walls 124a, 124b and configured to apply negative pressure to section 104b. Openings 105b of section 104b may be in fluid communication with channel 126 of tube 116b, such that when negative pressure is supplied to channel 126, the negative pressure may pull fluid, material, and/or other debris from target site 170 into channel 126 of tube 116b via openings 105b when section 104b is exposed to target site 170 (e.g., when section 104a is removed). Tube 116c may similarly include an outer wall 128a, an inner wall 128b, and a cylindrical lumen/channel 130 disposed between walls 128a, 128b and configured to apply negative pressure to section 104c. Openings 105c of section 104c may be in fluid communication with channel 130 of tube 116c, such that when negative pressure is supplied to channel 130, the negative pressure may pull fluid, material, and/or other debris from target site 170 into channel 130 of tube 116c via openings 105c when section 104c is exposed to target site 170 (e.g., when sections 104a and 104b are removed).

Tube 116d may include a wall 132 defining a central, cylindrical lumen/channel 134. Channel 134 extends from a proximal end of tube 116d, shown in FIG. 1D, to a distal end of tube 116d, shown in FIG. 1B. Central longitudinal axis A of EVAC device 100 may extend through central channel 134 of tube 116d. Similar to channels 122, 126, and 130, channel 134 may have open proximal and distal ends. Openings 105d of section 104d may be in fluid communication with channel 134 of tube 116d, such that when negative pressure is supplied to channel 134, the negative pressure may pull fluid, material, and/or other debris from target site 170 into channel 134 of tube 116d via openings 105d when section 104d is exposed to target site 170.

In some examples, an inner surface of each tube 116a, 116b, 116c may contact an outer surface of the adjacent tube 116b, 116c, 116d, respectively. Inner wall 120b of tube 116a may contact outer wall 124a of tube 116b, inner wall 124b of tube 116b may contact outer wall 128a of tube 116c, and inner wall 128b of tube 116c may contact wall 132 of tube 116d.

Each tube 116a, 116b, 116c may be configured to be movable over a remainder of tubes of tubular assembly 116. Each tube 116a, 116b, 116c may be configured to be movable over/along and relative to one or more of tubes 116b, 116c, 116d. For example, tube 116a may be configured to move proximally over/along and relative to tubes 116b, 116c, 116d to retract section 104a from target site 170, tube 116b may be configured to move proximally over/along and relative to tubes 116c, 116d to retract section 104b from target site 170, and tube 116c may be moved proximally over and relative to tube 116d to retract section 104d from target site 170. For example, wall 120b of tube 116a (or an inner surface of tube 116a) may slide along wall 124a of tube 116b (or an outer surface of tube 116b), wall 124b of tube 116b (or an inner surface of tube 116b) may slide along wall 128a of tube 116c (or an outer surface of tube 116c), and wall 128b tube 116c (or an inner surface of tube 116c) may slide along wall 132 of tube 116d (or an outer surface of tube 116d). For example, a lubricating material or an air gap may be present between adjacent tubes 116a, 116b, 116c, 116d to facilitate removal of one or more of the tubes.

In an alternative, inner walls 120b, 124b, and 128b may be omitted. Tubes 116a, 116b, 116c may include single tubular walls 120a, 124a, 128a. Walls 120a, 124a, 128a may be fixed to radially outermost portions of sections 104a, 104b, 104c, respectively. A space/gap between wall 120a and wall 124a may define channel 122. A space between wall 124a and wall 128a may define channel 126. A space between wall 128a and wall 132 may define channel 130. Because walls 120a, 124a, 128a may be fixed to radially outermost portions of sections 104a, 104b, 104c, channels 122, 126, 130 may supply suction to only sections 104a, 104b, 104c, respectively, and not to the other sections. Because of the spaces/gaps between walls 120a, 124a, 128a, 132, tubes 116a, 116b, 116c may be removable, as discussed above.

It will be appreciated that the number of tubes of tubular assembly 116 depends on the number of sections or layers in foam assembly 104. EVAC device 100 may include any number sections or layers (and any corresponding number of tubes), which may depend on, e.g., an expected length of the treatment and/or size of the wound or target site. For example, EVAC device 100 may include at least two sections or layers and at least two tubes.

EVAC device 100 may include an overtube 118 (FIG. 1A), which may extend over tubular assembly 116 and foam assembly 104 during delivery of EVAC device 100. Overtube 118 may facilitate movement of EVAC device 100 through a working channel of an endoscope or other medical device. For example, overtube 118 may compress foam assembly 104 into a lower profile during insertion to target site 170. Proximal movement of overtube 118 relative to EVAC device 100 may expand foam assembly 104 during positioning of foam assembly 104 within target site 170. For example, foam assembly 104 may have shape memory properties that cause foam assembly 104 to expand to a natural, relaxed shape after overtube 118 is retracted.

FIG. 1G shows an exemplary handle 102 of EVAC device 100 configured to couple to tubular assembly 116. Handle 102 is shown with a portion of a housing 103 removed to show inner features of handle 102. Housing 103 may define an inner cavity 101 to movably receive tubular assembly 116. A proximal end of housing 103 may define a first opening 107a, and a distal end of housing 103 may define a second opening 107b. Each of first opening 107a and second opening 107b may be in fluid communication with cavity 101. Tubular assembly 116 may slidably extend through first opening 107a and second opening 107b.

Handle 102 may include a first wheel 172 and a second wheel 174, and each wheel 172, 174 may be configured to rotate within a respective slot formed on opposite sides of housing 103. Each wheel 172, 174 may extend from outside the respective slot to an interior of cavity 101. Each wheel 172, 174 may contact an outer surface of tubular assembly 116 (e.g., an outer surface of an outermost tube, e.g., tube 116a). Handle 102 may further include a belt 176 extending from first wheel 172 (e.g., an axle A of first wheel 172) to second wheel 174 (e.g., an axle B of second wheel 174). Belt 176 may apply tension (an inward force) on first wheel 172 and second wheel 174 in order to maintain frictional engagement between each of first wheel 172 and second wheel 174 with tubular assembly 116.

Rotation of first wheel 172 and second wheel 174 in a forward or distal

direction (i.e., a counterclockwise direction of first wheel 172 in the view of FIG. 1G and a clockwise direction of second wheel 174 in the view of FIG. 1G) causes an outermost tube (e.g., tube 116a) of tubular assembly 116 to move in a backward or proximal direction relative to inner tubes (e.g., tube 116b, 116c, 116d) of tubular assembly 116. For example, friction between the outer surface of the outermost tube and first wheel 172 (and between the outer surface of the outermost tube and second wheel 174) causes first wheel 172 and second wheel 174 to grip onto the outer surface of the outermost tube to move the outermost tube proximally relative to the inner tubes. In some examples, wheels 172, 174 may be moved in the opposite direction to move the outermost tube relative to the inner tubes.

After the outermost tube (e.g., tube 116a) has been retracted proximally and removed, belt 176 may apply tension (inward force) to wheels 172, 174 so that wheels 172, 174 frictionally engage the tube that is now the outermost tube (e.g., tube 116b). In such a manner, subsequent (progressively inner) tubes may be moved proximally and removed, as discussed in the method below.

Although handle 102 is shown as having two wheels 172, 174, it will be appreciated that handle 102 may only include one wheel to proximally move tubes 116a, 116b, 116c of tubular assembly 116 over a remainder of tubes of tubular assembly 116. Wheels 172, 174 may be separately movable or may be configured to move together when an operator contacts one of wheels 172, 174. For example, an operator may contact and rotate one of wheels 172, 174, and the other of wheels 172, 174 may passively move due to a frictional force exerted by tubular assembly 116 on the other of wheels 172, 174.

An exemplary method of using EVAC device 100 will now be described. FIGS. 1A, 1E, and 1F illustrate a cross-sectional view of EVAC device 100 in use within a body lumen of a patient (e.g., a portion of the GI tract) to treat target site 170 (e.g., a wound, a leak, etc.). To position EVAC device 100 at target site 170, a user may insert an endoscope (or other medical device) into the patient via a natural orifice and position the endoscope proximate to target site 170. EVAC device 100 may be provided with overtube 118 positioned over foam assembly 104. Alternatively, the user may place overtube 118 over EVAC device 100. The user may insert overtube 118, along with EVAC device 100, into a working channel of the endoscope. The user may move overtube 118, along with EVAC device 100, distally through the working channel. With a distal portion of the endoscope positioned proximate to target site 170, the user may move overtube 118, along with EVAC device 100, distally out of the working channel and position foam assembly 104 within target site 170. Overtube 118 may then be moved proximally (retracted) to expand foam assembly 104 within target site 170, and expose section 104a of foam assembly 104 to target site 170. Overtube 118 may then be removed from the patient's body.

The user may then activate a vacuum source (not shown) to supply negative pressure to section 104a through channel 122 of tube 116a, which may pull fluid, material, and/or other debris from target site 170 into channel 122 of tube 116a and/or pull portions of target site 170 towards foam assembly 104. The endoscope may be removed, and the user may then leave foam assembly 104 and tubular assembly 116 positioned within the body of the patient for a suitable amount of time (e.g., 3-5 days).

As target site 170 heals, target site 170 may shrink and become smaller, as shown in FIG. 1E. The size of foam assembly 104 may no longer be suitable for the adjusted size of target site 170, and the user may remove section 104a of foam assembly 104 from EVAC device 100 to adequately match the adjusted size of target site 170. The user may first disconnect tube 116a of tubular assembly 116 from the vacuum source. The user may then retract tube 116a over tubes 116b, 116c, 116d to move section 104a proximally over sections 104b, 104c, 104d and over tubes 116b, 116c, 116d by, e.g., rotating first wheel 172 and second wheel 174 of handle 102 in a distal direction. Section 104a and tube 116a may be removed from the patient's body, while sections 104b, 104c, 104d and tubes 116b, 116c, 116d remain in target site 170, as shown in FIG. 1F. As discussed above, first wheel 172 and second wheel 174 of handle 102 may engage tube 116b following removal of tube 116a.

The user may then reconnect the vacuum source to tube 116b and activate the vacuum source to supply negative pressure to section 104b, which may pull fluid, material, and/or other debris from target site 170 into channel 126 of tube 116b and/or pull portions of target site 170 towards foam assembly 104. Sections 104b, 104c, 104d, and tubes 116b, 116c, 116d may be left within the body of the patient for a suitable amount of time (e.g., another 3-5 days).

As target site 170 continues to heal, target site 170 may continue to shrink and become smaller. The user may remove section 104b of foam assembly 104 so that foam assembly consumes less space within target site 170 and to allow target site 170 to continue to decrease in size as target site 170 heals. The user may disconnect tube 116b of tubular assembly 116 from the vacuum source. The user may then retract tube 116b over tubes 116c, 116d to move section 104b proximally over sections 104c, 104d and over tubes 116c, 116d. Section 104b and tube 116b may be removed from the patient's body, while sections 104c, 104d and tubes 116c, 116d remain in target site 170.

The user may repeat these steps until section 104d (an innermost section of foam assembly 104) is exposed to target site 170. The user may fully remove EVAC device 100 from the patient once treatment is complete by retracting tube 116d to remove section 104d from target site 170.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device comprising:
a tubular assembly including a plurality of tubes, wherein each tube of the plurality of tubes is configured to be coupled to a vacuum source to deliver negative pressure to a distal end of each respective tube of the plurality of tubes; and
a porous assembly including a plurality of sections and coupled to a distal end of the tubular assembly, wherein each section of the plurality of sections is coupled to a respective tube of the plurality of tubes.

2. The medical device of claim 1, wherein each tube of the plurality of tubes includes one or more walls defining a channel to deliver negative pressure to its respective section of the plurality of sections.

3. The medical device of claim 2, wherein a first channel of an innermost tube of the plurality of tubes extends from a proximal end to a distal end of the innermost tube, and wherein a central longitudinal axis of the medical device extends through the first channel.

4. The medical device of claim 3, wherein a second channel of an outermost tube of the plurality of tubes extends from a proximal end to a distal end of the outermost tube of the plurality of tubes, and wherein the second channel extends radially around the central longitudinal axis of the medical device.

5. The medical device of claim 2, wherein each section of the plurality of sections includes openings in fluid communication with the channel of the respective tube of the plurality of tubes, and wherein the openings of each section of the plurality of sections are independent of the openings of other sections of the plurality of sections.

6. The medical device of claim 2, wherein the channel of each of the plurality of tubes has an open proximal end and an open distal end, and wherein the open proximal end of the channel is coupled to the vacuum source and the open distal end is coupled to a proximal end of a respective section of the plurality of sections.

7. The medical device of claim 1, wherein the plurality of sections are configured to be removed individually from a target site within a body lumen.

8. The medical device of claim 1, wherein an outermost tube of the plurality of tubes is coupled to an outermost section of the plurality of sections, and wherein proximal movement of the outermost tube relative to other tubes of the plurality of tubes retracts the outermost section of the plurality of sections over a remainder of the plurality of sections.

9. The medical device of claim 1, wherein the plurality of tubes are configured to deliver negative pressure to only one section of the plurality of sections at a time.

10. The medical device of claim 1, wherein each section of the plurality of sections includes an inner surface and an outer surface, and wherein the outer surface of each section of the plurality of sections includes one or more of a course portion to collect exudates from a target site or a coating to prevent tissue ingrowth.

11. The medical device of claim 1, wherein each section of the plurality of sections includes a cap at a distalmost face of each section of the plurality of sections.

12. The medical device of claim 1, wherein a diameter of a distal end of each tube of the plurality of tubes aligns with a diameter of a proximal end of a respective section of the plurality of sections.

13. The medical device of claim 1, wherein the porous assembly includes at least four sections and the tubular assembly includes at least four tubes, and wherein a first section is coupled to a first tube, a second section is coupled to a second tube, a third section is coupled to a third tube, and a fourth section is coupled to a fourth tube.

14. The medical device of claim 13, wherein the first section is cylindrical, the second section extends around the first section, the third section extends around the second section, and the fourth section extends around the third section.

15. The medical device of claim 13, wherein the first tube is nested within the second tube, the second tube is nested within the third tube, and the third tube is nested within the fourth tube.

16. A medical device comprising:
a tubular assembly including at least two tubes, wherein each tube of the tubular assembly includes a channel, wherein each channel is configured to be coupled to a vacuum source to deliver negative pressure to a distal end of each tube independently of other channels; and
a porous assembly including at least two sections and coupled to a distal end of the tubular assembly, wherein each section of the porous assembly is coupled to a respective channel of the at least two tubes.

17. The medical device of claim 16, wherein an outermost tube of the at least two tubes is coupled to an outermost section of the at least two sections, and wherein proximal movement of the outermost tube relative to an innermost tube of the at least two tubes moves the outermost section of the at least two sections proximally over an innermost section of the at least two sections and over the innermost tube of the at least two tubes.

18. The medical device of claim 17, wherein a channel of the innermost tube of the at least two tubes is cylindrical, and wherein a channel of the outermost tube extends radially around a central longitudinal axis of the medical device.

19. A medical device comprising:
a tubular assembly including a plurality of tubes nested within one another, wherein each tube of the plurality of tubes is configured to be coupled to a vacuum source to deliver negative pressure to a distal end of each tube; and
a porous assembly including a plurality of sections nested within one another and coupled to a distal end of the tubular assembly, wherein a proximal end of each section of the plurality of sections is coupled to a distal end of a respective tube of the tubular assembly.

20. The medical device of claim 19, wherein each tube of the plurality of tubes is configured to independently deliver negative pressure to a respective section of the plurality of sections.
